(19) United States
(12) Patent Application Publication
(10) Pub. No.: US 2015/0174355 A1
(43) Pub. Date: Jun. 25, 2015

(54) FIXATION CONTROL FOR A PATIENT INTERFACE

(71) Applicant: KONINKLIJKE PHILIPS N.V., EINDHOVEN (NL)

(72) Inventors: Nicolaas Petrus Willard, Valkenswaard (NL); Joyce van Zanten, Waalre (NL); Willem Potze, Geldrop (NL); Cornelis Petrus Hendriks, Eindhoven (NL); Sima Asvadi, Eindhoven (NL); Rudolf Maria Jozef Voncken, Eindhoven (NL); Jacob Roger Haartsen, Eindhoven (NL); Mareike Klee, Straelen (DE)

(21) Appl. No.: 14/412,010

(22) PCT Filed: Jun. 24, 2013
(86) PCT No.: PCT/IB2013/055172

§ 371 Date: Dec. 30, 2014

Related U.S. Application Data
(60) Provisional application No. 61/667,479, filed on Jul. 3, 2012.

(51) Int. Cl. A61M 16/06; A61M 16/00

(52) U.S. Cl. CPC A61M 16/0644; A61M 16/0003; A61M 16/0051; A61M 16/0622; A61M 16/0683; A61M 2016/003; A61M 2016/0027; A61M 2205/18; A61M 2205/3306; A61M 2230/30

(57) ABSTRACT

The present invention relates to a detector, a patient interface and a patient interface system comprising this detector, and a method for preventing the formation of red marks due to the wearing of a patient interface (12) that is pressing against the skin of the patient (18) for a longer time. With the detector and via the method, the blood flow and/or the occlusion of the blood vessels of the patient (18) are monitored, and the assessed degrees are used as a basis to adjust the patient interface (12) in order to reduce the force pressing against the skin of the patient (18), such that the formation of red marks is prevented.

### FIELD OF THE INVENTION

The present invention relates to a detector for monitoring a degree of blood flow through at least one blood vessel, as well as to a cushion element with at least one sensor, a patient interface with at least one sensor, a patient interface comprising such a detector and a method for preventing a formation of red marks by a patient interface.

### BACKGROUND OF THE INVENTION

Patient interfaces, such as masks for covering the mouth and/or nose, are used for delivering gas to a patient. Such gases, like air, cleaned air, oxygen, or any modification of the latter, are submitted to the patient via the patient interface in a pressurized or unpressurized way.

For several chronic disorders and diseases, a long-term attachment of such a patient interface to a patient is necessary or at least advisable.

One non-limiting example for such a disease is obstructive sleep apnea or obstructive sleep apnea syndrome (OSA). OSA is usually caused by an obstruction of the upper airway. It is characterized by repetitive pauses in breathing during sleep and is usually associated with a reduction in blood oxygen saturation. These pauses in breathing, called apneas, typically last 20 to 40 seconds. The obstruction of the upper airway is usually caused by reduced muscle tonus of the body that occurs during sleep. The human airway is composed of walls of soft tissue which can collapse and thereby obstruct breathing during sleep. Tongue tissue moves towards the back of the throat during sleep and thereby blocks the air passages. OSA is therefore commonly accompanied with snoring.

Different invasive and non-invasive treatments for OSA are known. One of the most powerful non-invasive treatments is the usage of Continuous Positive Airway Pressure (CPAP) or Bi-Positive Airway Pressure (BiPAP) in which a patient interface, e.g. a face mask, is attached to a tube and a machine that blows pressurized gas, preferably air, into the patient interface and through the airway in order to keep it open. Positive air pressure is thus provided to a patient through a hose connected to a patient interface or respiratory interface, such as a face mask, that is worn by the patient. The afore-mentioned long-term use of the patient interface is the result, since the wearing of the patient interface takes place during the sleeping time of the patient.

Examples for patient interfaces are:

nasal masks, which fit over the nose and deliver gas through the nasal passages,

oral masks, which fit over the mouth and deliver gas through the mouth,

full face masks, which fit over both, the nose and the mouth, and deliver gas to both, total masks, which fit over the whole face, and

nasal pillows, which are regarded as masks as well within the scope of the present invention and which consist of small nasal inserts that deliver the gas directly to the nasal passages.

The patient interface is usually positioned on the patient's head using some kind of headgear. Wearing a patient interface can be uncomfortable, since for providing an airtight seal between the patient interface and the patient's face, the patient interface has to be worn tightly on the face.

In order to remedy that the wearing of the patient interface is uncomfortable, US 2008/0314390 A1 suggests to equip a facial mask with an automatically adjusting forehead support. This forehead support is adapted to be movable between two positions and includes a biasing mechanism that urges the forehead support in the second position. Thereby a more comfortable wearing of the mask shall be achieved.

However, as a part of such a patient interface being uncomfortable, the tightly wearing of the patient interface on the face may result also in pressure points and red marks once the patient interface is removed.

### SUMMARY OF THE INVENTION

It is an object of the present invention to provide a device and a method which provides a reduction of the formation of red marks, preferably avoiding them completely, and which therefore improves the comfort for a patient wearing a patient interface.

According to an aspect of the present invention, a patient interface system, with

a patient interface for providing a patient with a flow of gas, and

a detector for monitoring a degree of blood flow through at least one blood vessel of a patient is provided, with

at least one sensor, and

at least one monitoring unit,

wherein the at least one sensor is able to detect characteristic data of a blood flow through the at least one blood vessel and is connected to the at least one monitoring unit for transmitting the detected data to the at least one monitoring unit,
wherein the at least one monitoring unit is able to receive the data from the at least one sensor and is able to assess the degree of blood flow through the at least one blood vessel based on these data, and
wherein the at least one monitoring unit is able to provide a signal based on the assessed blood flow through the at least one blood vessel,

preferably wherein the monitoring unit is able to assess a degree of occlusion of the at least one blood vessel based on the assessed blood flow, and wherein the signal is based on the assessed degree of occlusion.

The term “occlusion” as used within the context of the present invention is to be understood as encompassing the complete occlusion or closure as well as preferably any level or degree of occlusion of the blood vessels if not specified or otherwise determined by the context.

The term “degree of occlusion” as used within the context of the present invention is to be understood as encompassing at least two stages, i.e. a completely opened and a completely occluded blood vessel, the latter meaning that no blood flows through the vessels. Preferably an imminent complete occlusion of the blood vessel and more preferably several degrees or levels of occlusion of the at least one blood vessel, selected from the range between no occlusion and complete occlusion of the at least one blood vessel is encompassed by this term.

The term “degree of blood flow” as used within the context of the present invention is to be understood as encompassing at least two stages, i.e. a maximum blood flow and no blood flow at all, the latter being the case if the blood vessels are completely occluded, for example. Preferably several degrees or levels of blood flow, selected from the range between the maximum blood flow and no blood flow at all is encompassed by this term.

Within the present invention, the terms “(degree of) occlusion” and “(degree of) blood flow” through the at least one blood vessel are often used synonym in explanations, since the blood flow corresponds to the degree of occlusion of the respective blood vessels.

The term “characteristic data of the blood flow” as used within the context of the present invention is to be understood as all data or information that can be detected from the blood flow itself and that may be influenced by the compression or occlusion of the blood vessels, such as but not limiting to pressure, density, volume of blood per time, particle density, sound, blood concentration in the surrounding tissue/skin etc.

The term “blood vessel” as used within the context of the present invention is to be understood as encompassing arteries and veins, wherein arteries are preferred within the context of the present invention. Within the scope of the present invention all blood vessels in the skin, under the skin or both are meant. However, within the present invention it is preferably referred to the blood vessels in the skin.

The term “signal” as used within the context of the present invention is to be understood as an acoustic, visual or electronic signal if not specified or otherwise determined by the context.

The term “gas” as used within the context of the present invention that is supplied to the patient is to be understood as any gas that is suitable to be supplied to a patient for the desired purpose, like treating OSA. Non limiting examples are air, cleaned air, oxygen and any modification of the latter. This gas may be supplied either pressurized or unpressurized.

The term “patient” as used within the context of the present invention is to be understood in a general way and not meant to be only limited to people suffering from an illness. Therefore, “patient” does also encompass people/users who use a patient interface, or the other devices and methods according to the present invention, for preventive measures with respect to their health or for other applications that are not directly related to illnesses or medical purposes.

According to another aspect of the present invention, a method for preventing a formation of red marks by a patient interface on a skin of a patient is provided, with the following steps:

attaching a patient interface to a patient by an attachment force, such that the patient interface contacts the skin of the patient,

assessing a degree of blood flow through at least one blood vessel of the patient, where the patient interface contacts the skin,

adjusting the attachment force such that a blood flow is present, preferably such that the blood flow is above a predetermined threshold, wherein the assessing the degree of blood flow is preferably realized by detecting characteristic data of the blood flow through the at least one blood vessel of the patient,

and the method preferably further comprises the step of assessing a degree of occlusion of the at least one blood vessel based on the assessed degree of blood flow.

Preferred embodiments of the invention are defined in the dependent claims.

Red marks are formed if too much pressure is applied to the skin and if the blood vessels, especially the arteries in the pressed area get occluded, meaning narrowed or closed, such that the amount of blood delivered to the tissue, especially the skin tissue around the arteries is too low. This pressure exerted by the patient interface may result from different or several parts of the patient interface, including the headgear, the cushion or pillow(s) and/or the forehead support, for example. When the arteries get narrowed, the blood flow is still present and can be detected up until the point to where the blood vessel or artery is completely occluded or closed. This will be the case when the applied pressure exceeds the systolic blood pressure in those arteries. However, even in the case of partially occluded arteries, i.e. narrowed arteries, the amount of blood delivered to the tissue around the arteries can be low enough to result in a formation of red marks. Accordingly, an occlusion, that is to say a complete or an imminent closure may lead to the formation of red marks and should therefore be avoided.

Considering the possible variations in skin parameters for one patient due to the long-term wearing of a patient interface, or also between different patients, like variations in blood pressure, tightening force of the headgear and the pressure distribution over the patient interface as well as the elasticity of the skin of the patient, a static system of a patient interface worn by a patient will almost inevitably result in the afore-mentioned formation of red marks on the patient's skin.

With the aid of the afore-mentioned detector, the degree of blood flow and also the degree of occlusion of the at least one blood vessel can be monitored. The data acquired from the at least one sensor can further be used to provide a signal by the monitoring unit of the detector. This signal can either be a continuous signal corresponding to the degree of blood flow through the at least one blood vessel or the corresponding degree of occlusion, for example. Alternatively, this signal can be dependent on a predetermined threshold. In the latter case, a signal is only provided if the degree of the blood flow or the degree of occlusion of the at least one blood vessel passes this threshold. This may be the case when the degree of the blood flow falls under the predetermined threshold, for example. In a concrete example the sensor detects the absence of the blood flow, for example because the blood vessels are completely occluded, and then provides a single signal, e.g. in form of a sound. Based on this signal, a headgear of the patient interface may be adjusted accordingly, e.g. by the patient, thereby reducing the pressure of the patient interface on the skin such that a blood flow reoccurs in the at least one blood vessel.

With respect to the method of the present invention, this can also be explained in that way that the attachment force acting on the patient interface, e.g. by a headgear, and accordingly on the skin of the patient, is adjusted such that the blood flow is still present, and preferably above a predetermined threshold. The result is that the amount of blood flowing through the at least one blood vessel is such that at least the formation of red marks is prevented.

The number of sensors used can depend on the points on the skin of a patient to be monitored such that in a preferred embodiment, the formation of red marks is avoided completely. The monitoring unit can either be one monitoring unit per sensor or may also be one monitoring unit for several or even all sensors.

Preferably, the degree of blood flow through the at least one blood vessel, or the degree of occlusion of the at least one blood vessel based on the degree of blood flow, encompasses more than two stages. As a result, not only the complete closure or occlusion of the at least one blood vessel may be detected but also at least the imminent closure of the blood vessels is detectable in order to prevent the formation of red marks at an earlier stage. Using these degrees it is possible to not only have one certain incident leading to a single signal, like the complete occlusion or closure of the blood vessels, but also to identify different stages of the occlusion of the at least one blood vessel by a signal, for example. Accordingly, measures for adjusting the attachment force may be taken at an earlier stage before the complete occlusion of the at least one blood vessel occurs. This is especially beneficial if it is desired to maintain the pressure of the patient interface on the patient's skin at a certain level or within a certain pressure range such that the blood flow is only reduced to a certain amount. Thereby a reduction of the blood flow to an amount that it is at least half of its normal value is possible, for example.

According to another embodiment of the detector, the sensor comprises a light source for transmitting light into the skin and an optical sensor for detecting the reflected light. The blood flow in the blood vessels, preferably in the skin, can be measured by the light absorption or reflection of particles in the blood, like red blood cells. The amount of blood flowing in the blood vessels corresponds to the amount of red blood cells in the blood vessels and accordingly to the concentration of the red blood cells in the skin. Since the amount of blood flowing through the blood vessels depends on the degree of occlusion of the blood vessels, this detectable red blood cell amount or concentration can be used to assess the mentioned degree of occlusion. This can be measured by the optical sensor either via the light absorption or the light reflection by the red blood cells of the light transmitted into the skin. One example for detecting the concentration in the skin would be by monitoring the color of the skin with the aid of the light source and optical sensor. If the concentration of red blood cells in the skin is reduced the color of the skin shifts from red to white. The detected color may be compared to a threshold and a signal may be given if this threshold is passed. The threshold may be either predetermined or determined individually, e.g. for every patient, on a daily basis or even regularly by the device itself. By measuring the concentration of the red blood cells within the blood vessels with respect to time via the optical sensor, a detection of the oscillation of the blood flow may be possible. This oscillation is the result of the pressure differences due to the pulsating contractions of the beating heart. A narrowing of the blood vessels, especially the arteries, leads to changes in this detected oscillation, e.g. in the amplitude, such that the degree of occlusion of the blood vessels can be assessed based on the (change in) detected oscillation. The term “optical sensor” as used within the context of the present invention is to be understood as not only including light sensors suitable for the mere detection of the general intensity of the light, but also including optical sensors that are at least capable of detecting the intensities of light with a certain wavelength or a combination of certain wavelengths, meaning individual colors.

According to another embodiment of the detector, the sensor comprises a sound detector. If blood vessels, especially arteries, are closed, the almost linear flow of the blood through the blood vessels shifts to a turbulent flow at a certain degree of occlusion. At such a degree the complete closure of the blood vessel is imminent. This turbulence of the blood flow results in certain pulsating sounds called Korotkoff sounds. Therefore, this characteristic sound is an indicator for the imminent closure of the blood vessels, which is why this imminent closure may be detected by this sound and the aforementioned measures, like releasing the force of the patient interface pressing on the skin of the patient, may be taken. This may be done by the attachment force being released until the detected sounds disappear, meaning until there is no turbulent blood flow anymore, for example. These sounds may be detected by a sound detector. Such a sound detector may be either a microphone being directly a part of the detector of the present invention or may also be some kind of sound transmission element, like a tube or any other suitable device that leads to an external microphone. The microphone, receiving the sound from the arteries through the skin either directly or indirectly, converts this sound into an electronic signal.

According to another embodiment of the detector, the sensor comprises a pressure sensor. Such a pressure sensor can detect the oscillations or pulsations in the blood flow. Therefore, this pressure sensor gets into contact, either directly or indirectly, via the skin and/or the underlying soft tissue with the arteries thereby detecting the alternating pressure in the arteries as a result of the pulsating heart contractions. Accordingly, it is not necessary for such a sensor to be calibrated to measure the (accurate) blood pressure. Such a sensor only needs to detect pressure differences. However, embodiments that allow also an accurate blood pressure measurement shall also be regarded as being within the scope of the present invention. Due to the pulsation in the blood flow, such a sensor provides an oscillating signal. Such an oscillating signal is an indication for the presence of the blood flow. If an oscillation is no longer detected, the blood flow through the blood vessels stopped and the formation of red marks will occur. In order to prevent the formation of red marks, the aforementioned force of the patient interface pressing against the skin of the patient may be adjusted based on the detected oscillation. If the oscillation is no longer detected, the force may be lowered until an oscillation is detected again, for example. Since the oscillation parameters correspond to the degree of occlusion of the blood vessels, this degree can be assessed based on these parameters, e.g. based on the amplitude.

According to another embodiment of the detector of the present invention, the detector further comprises an evaluation unit, wherein the evaluation unit is designed to receive and to process the signal from the monitoring unit and wherein the evaluation unit is able to control other devices via a control signal based on these processed signals. The evaluation unit is able to monitor the signal provided by the monitoring unit and, for example, able to compare this signal with the afore-mentioned threshold. If the assessed degree of blood flow or the assessed degree of occlusion of the blood vessels passes this predetermined threshold, the evaluation unit may control other devices based on this incident. One example for such a device may be a sound generator of any kind, giving an alarm to the patient. Based on this alarm, the patient knows that the attachment force of the patient interface on his/her face is too strong and that he/she should now lower the attachment force via an adjustment mechanism of the patient interface in order to prevent the formation of red marks. Another possibility for such a device may be an automatic adjustment mechanism acting on the headgear of the patient interface. Such an automatic adjustment mechanism may then, controlled by the evaluation unit, loosen, or also tighten the headgear. Thereby the force of the attachment of the patient interface to the patient may be reduced or also increased. The latter may be desired, if the detector detects that the assessed degree of occlusion of the blood vessels is far from the threshold and that a stronger attachment is possible in order to prevent a leakage of the gas supplied to the patient via the patient interface without the formation of red marks.

According to another aspect of the present invention, a cushion element for a patient interface is provided, with at least one sensor that is able to detect characteristic data of a blood flow through at least one blood vessel. Further, according to another aspect of the present invention, a patient interface for providing a patient with a flow of gas is provided, with at least one sensor that is able to detect characteristic data of a blood flow through at least one blood vessel.

The term “cushion element” as used within the context of the present invention is to be understood as a part of the patient interface normally comprising a soft material and being arranged on the side of the patient interface that is directed to and is getting in contact with the patient when the patient interface is worn by the patient. Thereby this cushion element provides an airtight seal and/or makes the wearing of the patient interface more comfortable for the patient. Therefore, the cushion element may be a part of the mask part which provides the gas to the patient or any other part of the patient interface that gets in contact with the patient, like a forehead support, a headgear etc. Accordingly, other words for describing this “cushion element” would be cushion, seal, pad, pillow, or the like.

The at least one sensor in this cushion element or patient interface is preferably the at least one sensor of a detector as mentioned before. Such a cushion element with the at least one sensor is an element of a patient interface that can be replaced. Therefore, comprising just a cushion element with the at least one sensor provides the possibility to exchange the cushion element in the case of a defect, for example, or to include a cushion element with the at least one sensor in a patient interface that does not comprise such an at least one sensor or detector in order to upgrade the whole system. Aside from comprising just the at least one sensor, the cushion element may also comprise at least one detector according the embodiments as mentioned before. A patient interface with just the sensor could also be an element of a system, wherein at least the monitoring unit of the detector stays in the system while the patient interface with the at least one sensor may get easily replaced.

According to an embodiment of the cushion element, the at least one sensor comprises a light source for transmitting light into the skin and an optical sensor for detecting the reflected light, a sound detector or a pressure sensor. According to an embodiment of the patient interface, the at least one sensor comprises a light source for transmitting light into the skin and an optical sensor for detecting the reflected light, a sound detector or a pressure sensor.

According to another embodiment of the cushion element, the at least one sensor is located in an area of the cushion element that corresponds to an area on the skin of a patient that is prone to a formation of red marks when the cushion element is applied to the patient. According to another embodiment of the patient interface, the at least one sensor is located in an area of the patient interface that corresponds to an area on the skin of a patient that is prone to a formation of red marks when the patient interface is applied to the patient. Preferred examples for such areas on the skin of a patient are the area around the nasal bone of a patient, the so called nose-bridge, the area between the nose and the cheeks, the chin area, and the forehead. However, the at least one sensor might be located in other areas as well. Based on the respective type of patient interface, not all of the afore-mentioned areas might be affected or even accessible. For example, in a patient interface with a full face mask only the nose-bridge, the area between cheeks and nose and the chin may be affected. Therefore, in an exemplary embodiment, four sensors would be used (one for the nose-bridge, one for the chin, and two for the area between cheek and nose, that is to say one for each side of the nose). However, aside from using just one sensor in the nose-bridge and the chin area, two sensors may be used respectively to avoid wrong results from the sensors due to the patient lying on one side of his/her face during sleep. In a total mask, especially the forehead and the chin would be affected, therefore one or two sensors for the forehead area and one or two for the chin would be used. In nasal masks, especially the headgear pressing against the skin affects the cheeks. Therefore, one sensor for each side would be used. Aside from these specific exemplary embodiments, any other suitable or necessary positioning of the sensors may be used, whereby the respective areas may be either less prone to a formation of red marks or may turn out to be prone to a formation of red marks.

According to another aspect of the present invention, a patient interface for providing a patient with a flow of gas, with a detector of anyone of the embodiments as mentioned before is provided. Providing such a patient interface gives the benefits as mentioned before to this whole device. The detector may in such a patient interface be arranged in or on the patient interface depending on the suitable position of the sensors of the detector as mentioned before.

According to another embodiment of the patient interface, the latter comprises:

a headgear for attaching the patient interface to a patient, and

at least one adjustment mechanism for regulation of a force for attaching the patient interface to the patient with the headgear,

wherein the regulation of the force is based on the signal provided by the detector. By the adjustment mechanism, a regulation of the force is possible, for example by the user. This can be done based on the signal provided by the detector, which preferably will occur if a closure of the at least one blood vessel is detected or a predetermined threshold for the degree of the occlusion of the blood vessels is passed.

According to another embodiment of the patient interface, the regulation of the force is controlled by the signal provided by the detector resulting in an automatic regulation of the force. In this manner, the patient interface uses the monitored blood flow, that is to say the assessed degree of blood flow or the assessed degree of the occlusion of the at least one blood vessel to modify the force of the patient interface pressing on the skin, such that no interaction of the patient with the patient interface is necessary. The whole patient interface then works automatically and adjusts the force in accordance with the embodiments as mentioned before. This means that either the force via which the patient interface is pressing on the skin of the patient is lowered, if the blood flow stops or falls under a predetermined threshold, or the force is increased, if the degree of the blood flow is (far) away from the threshold and the occurrence of a leakage of the gas supplied to the patient shall be avoided.

Aside from a patient interface comparable to the embodiments as mentioned before, the patient interface system may further comprise devices for supplying the air or pressurized gas to the patient, the supply of the gas, a power supply, or the like. The detector may either be a part of the patient interface or may be arranged at any other suitable position in the patient interface system. Therefore, the detector may be arranged in the patient interface system as a compact device, including sensor and monitoring unit, or may as well be arranged with the sensor and the monitoring unit being separated. For example, an embodiment where the sensor is arranged at the patient interface and the monitoring unit is arranged at an external device, which is a part of the patient interface system, like the ones mentioned before, would be possible.

According to another aspect of the present invention, a use of a detector of anyone of the embodiments as mentioned before for preventing an occlusion of at least one blood vessel of a patient due to wearing a patient interface for supplying gas to the patient is provided.

According to another aspect of the present invention, a headgear assembly for a patient interface is provided, with at least one sensor that is able to detect characteristic data of a blood flow through at least one blood vessel.

### BRIEF DESCRIPTION OF THE DRAWINGS

These and other aspects of the invention will be apparent from and elucidated with reference to the embodiments described hereinafter. In the following drawings

FIG. 1 shows a schematic perspective view of a patient wearing a patient interface according to the present invention,

FIG. 2 shows a schematic perspective view of the patient interface with detectors according to the present invention,

FIG. 3 shows a schematic representation of the detector according to the present invention,

FIG. 4 shows a schematic side view of a patient wearing a patient interface according to the present invention, and

FIG. 5 shows a schematic side view of a patient wearing a patient interface according to another embodiment of the present invention.

### DETAILED DESCRIPTION OF THE INVENTION

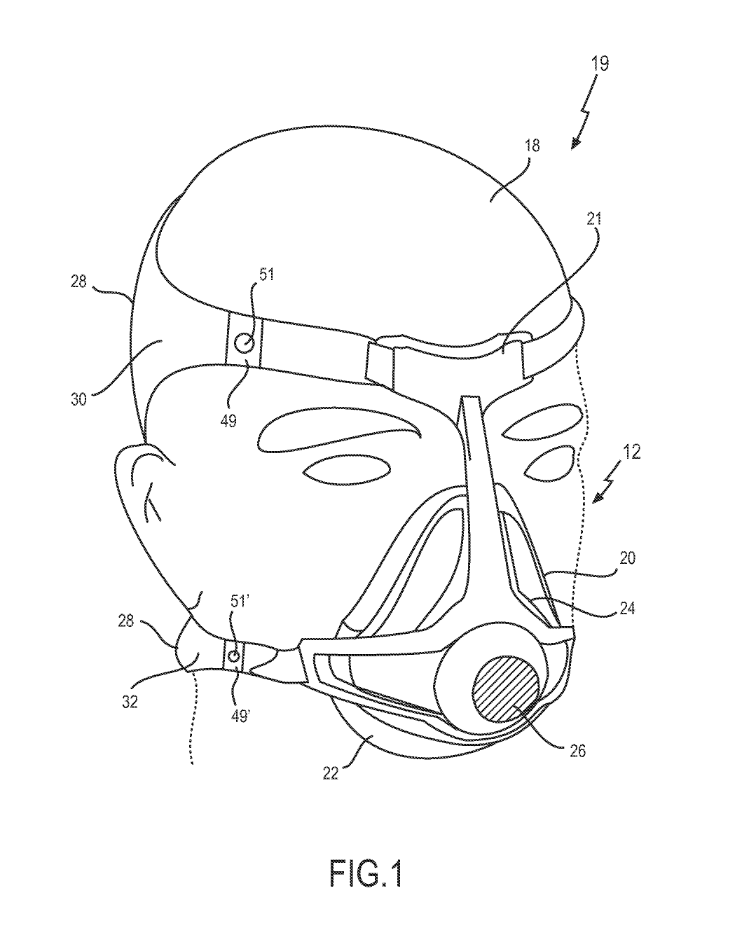

An embodiment of a detector according to the present invention is shown throughout and described with the help of FIGS. 1 to 5 and is designated in its entirety by the reference numeral 10. Further, patient interfaces according to the present invention comprising the detector 10 are shown throughout and described with the help of FIGS. 1 to 5 and are designated in their entirety by the reference numerals 12, 14 and 16, depending on the respective embodiment.

The patient interface 12, shown in FIG. 1, is worn by a patient 18. In this certain embodiment, the patient interface 12 comprises a full face mask 20, covering the mouth and nose of the patient 18, and a forehead support 21. The full face mask 20 comprises a cushion 22 and a shell 24. The cushion 22 is arranged on the shell 24 on that side that is directed to the face of the patient 18 in order to make the wearing of the full face mask 20 and of the patient interface 12 in general more comfortable and especially to provide an airtight seal of the full face mask 20 on the patient's face. For this, the cushion 22 is comprised of a soft material, like silicone rubber or any other rubber or suitable elastic material. On the opposite side directing away from the patient's face, the shell 24 comprises a connector 26. Via this connector 26 the patient interface 12 is able to be connected to a hose (not shown) via which the (pressurized) gas can be submitted to the patient 18. Together with a supply for the gas (not shown), the hose and the patient interface 12 form a patient interface system 19.

For attaching the full face mask 20 on the patient 18, the patient interface 12 comprises further a headgear 28. This headgear 28 is in this certain embodiment of FIG. 1 comprised of two straps 30 and 32 circumventing the head of the patient 18, thereby attaching the patient interface 12 with a certain attachment force on the patient's face.

Because of this attachment force, the patient interface 12, especially the cushion 22 presses against the skin of the patient 18 that lies under said cushion 22. This may result in a formation of red marks due to the significant reduction or stop of the blood flow in the blood vessels, especially the arteries within the skin of the patient in this area.

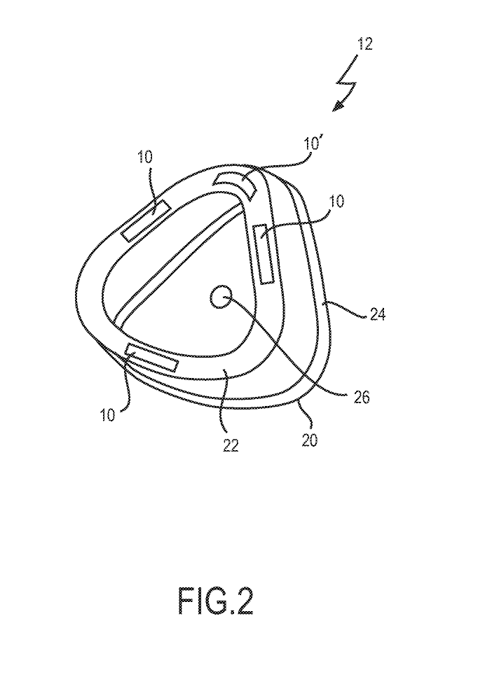

In order to prevent the formation of red marks on the skin of patient 18, the patient interface comprises detectors 10. These are shown in FIG. 2, for example.

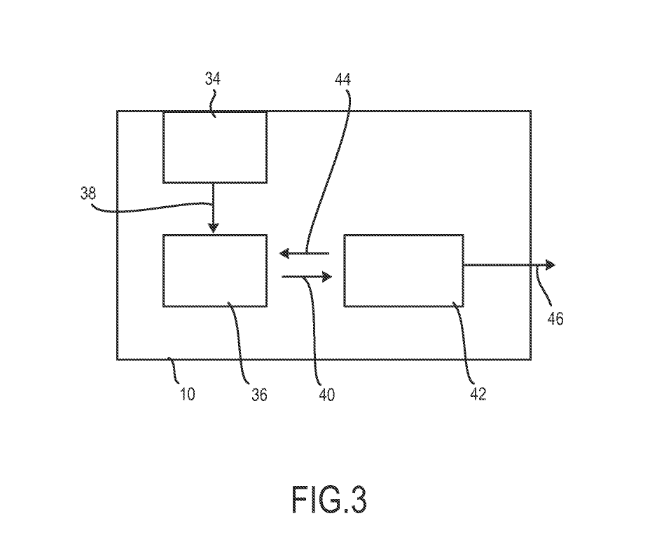

FIG. 2 shows the patient interface 12 with the cushion 22 and the shell 24 including the connector 26, but without the headgear 28 for clarity reasons. The detectors 10 are arranged in the cushion 22 of the patient interface 12. In this certain embodiment, the patient interface 12 comprises four detectors 10. Although this is to be understood merely as an example, since the patient interface as described within the present invention may comprise any suitable number of detectors each located at any suitable position, at least a detector 10′ located in the region corresponding to the upper end of the nasal bone, in the so called nose bridge area, is preferred in this embodiment, since this area is particularly sensitive for the formation of red marks. Further detectors are preferably arranged in the area that corresponds to the cheeks and the chin as shown in FIG. 2. The detector 10 is able to monitor the degree of blood flow through at least one blood vessel of the patient 18. As shown schematically in FIG. 3, this detector 10 therefore comprises a sensor 34 and a monitoring unit 36. Aside from this shown embodiment, other embodiments, where the cushion 22 comprises just the sensors 34 and not the whole detectors 10, for example in the positions shown for the detectors 10, lie within the scope of the present invention. The monitoring unit(s) 36 may then be either arranged at another part of the patient interface 12 or also at another part of the whole patient interface system 19.

The sensor 34 is able to detect characteristic data of the blood flow of the patient 18, which may be used as indicators for the degree of blood flow through the at least one blood vessel and in the consequence also for the degree of the occlusion of the at least one blood vessel. These characteristic data can be present in the form of the concentration of red blood cells or other particles, the variation of the pressure within the blood vessels or in the form of the sound of the blood that flows through the blood vessels, for example. Accordingly, this sensor 34 may comprise a light source, like an LED or a laser, and an optical sensor. With such an arrangement, light can be transmitted through the skin into the respective blood vessels where it is partly absorbed and partly reflected. Depending on the concentration of particles, like the red blood cells in these blood vessels, the amount of reflected light varies with the amount or concentration of these cells. Accordingly, the amount of blood flowing through the blood vessels per time can be detected by the optical sensor. By using light with a defined wavelength, this can also be done selectively by only detecting the red blood cells, for example. Also, a detection of the color of the skin is possible, which changes do to the concentration of red blood cells within the skin. The light source and the optical sensor may be arranged on the patient interface in any suitable way. Therefore, an arrangement of either one of them or both of them in direct contact with the skin is as possible as an arrangement wherein the light transmits through the cushion 22, for example, provided that the latter comprises a suitable transparency.

Alternatively, the sensor 34 may be a sound detector. A sound detector may either be a microphone arranged directly within this sensor 34 or may be a microphone indirectly connected to this sensor 34 via some kind of sound transmission. Such a sound transmission may be realized for example by tubes, sound transmitting materials or the like. Such a microphone may then, either directly or indirectly, receive the sound that the blood flow is causing when the blood comprises a turbulent flow through the arteries of the patient 18. Such a turbulent flow occurs when the blood vessels, e.g. the arteries, are occluded to a certain degree/level, if the pressure acting on the arteries is higher than the diastolic pressure in these arteries. These sounds resulting from the turbulent flow are also known as Korotkoff sounds. If the pressure acting on the blood vessels is high enough to produce these sounds a complete closure of the blood vessels is imminent. Accordingly, the mere presence of this sound may be an indicator to adjust the headgear 28, i.e. lower the attachment force of the patient interface 12 on the face of the patient 18.

As another alternative the sensor 34 may comprise a pressure sensor. Such a pressure sensor may directly detect the variation in the blood pressure of the respective arteries under the sensor 34. These variations in blood pressure do not need to be detected by a calibrated pressure sensor since the absolute blood pressure values are of less importance for this application. The pressure sensor merely needs to detect a difference in the pressure in the blood vessels as a result of the contractions of the heart and the pulsating blood flow. In this way the pressure sensor may detect an oscillating or pulsating variation in the pressure that corresponds to the pulsating blood flow through the blood vessels. Presence of the oscillation indicates the existence of the blood flow, whereas the absence of the oscillation may indicate that the blood flow is inhibited, i.e. that the blood vessels are occluded or closed. Also, the magnitude of the oscillation, e.g. via the amplitude, may indicate the degree of blood flow and accordingly the degree of occlusion of the blood vessels.

Independent of the special kind of the sensor 34, the latter is further able to transmit the detected data to the monitoring unit 36. This is indicated by an arrow 38. Accordingly, the monitoring unit 36 may receive this transmitted data from the sensor 34 as indicated by arrow 38. Further, the monitoring unit 36 may assess the degree of blood flow through the blood vessels, or even the degree of occlusion of the blood vessels based on the data received from the sensor 34. Based on this detected data, the monitoring unit 36 may provide a signal. This is indicated by an arrow 40. This signal 40 may be provided if the blood flow is no longer detected and accordingly a complete occlusion of the at least one blood vessel occurred. This may be the case if the patient interface 12 is pressing so strong against the face of the patient 18 that the blood flow within the arteries in the patient's skin stops. This would inevitably result in the afore-mentioned formation of red marks. Alternatively, the monitoring unit 36 may provide this signal 40 if a predetermined threshold is passed, either by the degree of blood flow falling below this threshold or exceeding this threshold, for example. Further, the monitoring unit 36 may provide a continuous signal 40 indicating the amount of blood per time, for example. The signal 40 provided by the monitoring unit 36 may further be a processed continuous signal that provides information about the degree of blood flow through the blood vessels or the degree of occlusion of the blood vessels, for example based on the detected amount of blood flowing through the blood vessels per time.

Further, the detector 10 may comprise an evaluation unit 42. This evaluation unit 42 is able or designed to receive the signal 40 of the monitoring unit 36. Further, the evaluation unit 42 may be able to send back another signal to the monitoring unit 36. This is indicated by another arrow 44. This signal 44 may be for example a control signal, a feedback signal or the like. The evaluation unit 42 may further process the signal 40 of the monitoring unit 36. Especially in the case where the monitoring unit 36 is not designed to compare the signal with a certain threshold, the evaluation unit 42 may process the data submitted via the signal 40. In processing the data of the signal 40, the evaluation unit 42 itself may compare the data with a certain threshold. This threshold may either be realized by a single value that merely shall not be passed or can also be designed as a threshold range in which the degree of blood flow or degree of occlusion of the blood vessels, monitored based on the data transmitted via the signal 40, shall be kept. One possible threshold would be that the amount of blood flowing through the blood vessels per time is zero. In this case, the blood flow within the arteries in the skin of the patient 18 stopped. This would result in a situation where the formation of red marks on the skin of the patient 18 occurs. An alternative and preferred threshold would be to keep the occlusion of the blood vessels and therefore the blood flow at a degree that the blood flowing within the arteries is approximately half to the normal blood flow that occurs when no patient interface is worn by the patient 18. This can for example be determined by the detector 10 registering the amount of blood flowing in the blood vessels per time in a loose attachment of the patient interface 12 to the patient 18 and then setting the threshold for this characteristic parameter at a value that is half of the afore-mentioned normal blood flow. This setting can either be done manually or preferably automatically by the detector 10.

Although the monitoring unit 36 and the evaluation unit 42 are shown here in FIG. 3 as separate parts, that may also be arranged at completely different locations within the patient interface (system), it goes without saying that the monitoring unit 36 and the evaluation unit 42, as well as the sensor 34, may be comprised as a single part via a single circuit or other electronic layout dependent on the technical possibilities and the desired design of such a detector 10. Also, aside from the embodiment shown, it is further possible to combine several evaluation units 42 to one respective combined evaluation unit in a patient interface that receives and/or processes the signals 40 from several monitoring units 36. Accordingly, the same works with respect to the monitoring units 36 in a patient interface which can also be designed as a combined monitoring unit receiving all the signals 38 from the sensors 34.

If in any of the afore-mentioned cases and embodiments the predetermined threshold is passed, or the threshold area is left, the evaluation unit 42 is able to control other devices as a result of this passing of the threshold. This control is indicated by an arrow 46. Non-limiting examples for a control are the control of a sound generator that generates a predetermined alarm when the threshold is passed. Thereby the patient 18 is made aware of the fact that the patient interface 12 is pressing too strong against his/her skin of the face. Based on this, the patient 18 may then readjust the patient interface 12. This can be done by adjusting the headgear 28. For this, the headgear 28 of the patient interface 12 may comprise an adjustment mechanism 49. This is shown in the embodiment of FIG. 1. Here, the adjustment mechanism 49 is exemplary shown for the strap 30. This adjustment mechanism 49 allows for a modification of the length of the strap 30 and, therefore, for an adjustment of the force via which the patient interface 12 is pressing against the face of the patient 18. An example for an adjustment mechanism 49 comprising an easy way for the patient 18 to lower the force is, for example, an adjustment mechanism 49 that comprises a button 51. Via this button 51, the patient 18 may be able to loosen the fit of the patient interface 12 on his/her face via a single push of the button 51. This is especially beneficial in the case of OSA considering that the patient interface 12 is worn during sleeping time. This easy way of releasing the force allows the patient 18 to quickly proceed this way and get back to sleep as soon as possible. By pushing the button 51, it is either possible that the strap 30 is completely released as long as the button 51 is pushed or that a predefined length of the strap 30 is released with each push. Although described for the strap 30, it goes without mentioning that the strap 32 may as well comprise an adjustment mechanism 49′. This adjustment mechanism 49′ may also comprise a button 51′.

Another example for a control by the evaluation unit 42 would be a direct control of any mechanism on the headgear 28 as will be described in the context of the following embodiments. Therein, the same elements with respect to the patient interface 12 are designated by the same reference numerals.

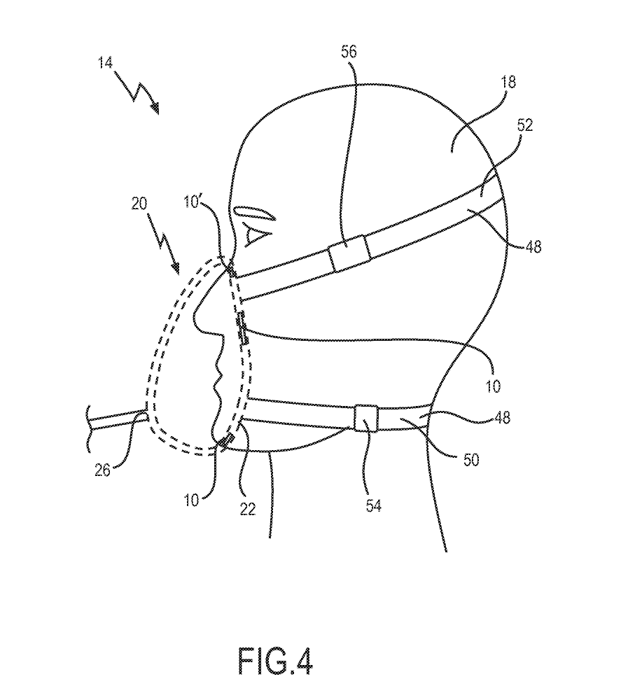

FIG. 4 shows another embodiment of a patient interface according to the present invention, i.e. the patient interface 14. This patient interface 14 also comprises a headgear 48. This headgear 48 comprises two sets of straps 50 and 52. Each of these straps 50 or 52 comprises an adjustment mechanism 54 and 56. In contrast to the embodiment of the patient interface 12, the present adjustment mechanisms 54 and 56 are designed such that they are able to adjust the length of the straps 50 and 52 via small electric motors (not shown in detail). These adjustment mechanisms 54 and 56 may be controlled via certain signals sent by the detector 10. Preferably, these signals are sent by the evaluation unit 42 as indicated by the arrow 46 in FIG. 3. The transmission of the signals may be realized via cables that are included in the mask and/or straps material (therefore not shown here). Alternatively, this transmission may be realized in a wireless way.

If the detector 10 detects a passing of a certain predetermined threshold in the degree of blood flow through the at least one blood vessel or the degree of occlusion of the at least one blood vessel as mentioned before, it may control the small electric motors within the automatic adjustment mechanisms 54 and 56. If the degree of occlusion is too high (or the degree of blood flow is too low), this control would be such that the straps 50 and/or 52 are loosened. Thereby the pressure of the patient interface 14 pressing on the face of the patient 18 is reduced. The result is that the blood may now again flow sufficiently within the arteries in the patient's face and the formation of red marks is avoided. This may either be realized by the detector 10 controlling the adjustment mechanisms 54 and/or 56 via a certain signal giving a predefined additional length to the straps 50 and/or 52, or by the detector 10 controlling the adjustment mechanisms 54 and/or 56 such that they are lengthening the straps 50 and/or 52 until a sufficient blood flow is detected again by the respective detector 10, for example. If, like in the present examples of the patient interfaces 12, 14 and 16, several detectors 10 or at least sensors 34 are present, such control of the adjustment of the headgear 48 can be realized either by a simultaneous control of the adjustment mechanisms 54 and 56 or also by a control of that adjustment mechanism 54 or 56 that would result in the reduction of the pressing force in that area where the respective detector 10 or sensor 34 had just detected the deficiency in the blood flow. For example, if the detector 10′ detects a stop of the blood flow within the skin of the patient 18, it may be designed such that it only controls the adjustment mechanism 56. Thereby only the strap 52 is loosened. Accordingly, the adjustment mechanism 54 is not activated by this detector 10′ and the length of the strap 50 is kept as it is.

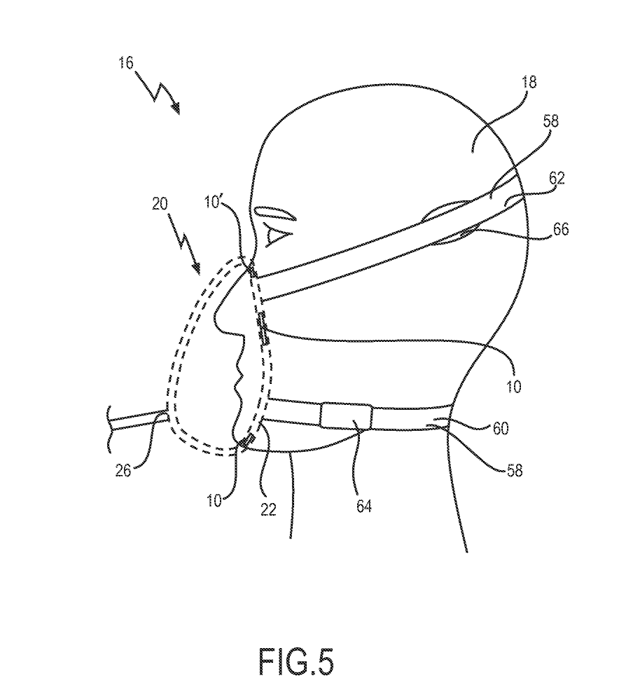

FIG. 5 shows another embodiment of a patient interface 16 according to the present invention. In this embodiment of the patient interface 16 the latter also comprises a headgear 58. This headgear 58 comprises two straps 60 and 62. Each strap 60 and 62 comprises a respective adjustment mechanism 64 and 66.

The adjustment mechanism 64 is realized in this special embodiment as an electroactive polymer 68. Such a polymer 68 can be changed in size, e.g. in its length, by the application of an electrical field to this polymer 68. Such an electrical field may be sent or at least controlled by the detector 10, especially by the corresponding evaluation unit 42 as shown via the arrow 46 in FIG. 3. If this is not done directly by the detector 10, the latter may control a source of an electrical field in order to increase or decrease this field. Accordingly, the length of the strap 60 in this embodiment is dependent on the length of the included adjustment mechanism 64 in form of the electroactive polymer 68. Therefore, this electroactive polymer 68 may replace a part of the strap 60 or may be designed such that it is placed on the strap 60 in a certain area and fixed with its opposing ends thereon. Thereby, the electroactive polymer 68 is able to shorten the length of the strap 60 depending on the length of the electroactive polymer 68, whereas the maximum length is given by the strap 60 itself.

Alternatively and shown here within the same embodiment of the patient interface 16, the adjustment mechanism 66 may be realized by an expandable body 70, like a balloon. Such an expandable body 70 may be in- or deflated by a controllable valve (not shown). Such a control may be realized again by a respective detector 10, preferably by the evaluation unit 42 as indicated by the arrow 46 in FIG. 3. This expandable body 70 is in this exemplary embodiment arranged on the side of the strap 62 that is directed to the head of the patient 18. Thereby, the expandable body 70 lies between the head of the patient 18 and the strap 62. Since it is possible that the volume of the expandable body 70 is changed in a continuous way, the circumference that is partially covered by the strap 62 may also change continuously. The result is that the force via which the patient interface 16 is pressing against the skin of the face of the patient 18 can be alternated in the same continuous way. This can be realized for example by the controllable valve being set to a position where a fluid medium can flow into the expandable body 70 resulting in an expansion of the expandable body 70 and accordingly a tightening of the strap 62, thereby increasing the force of the patient interface 16 on the skin of the patient 18. Alternatively, the valve can be controlled such that it is in a position that this fluid may flow out of the expandable body 70, thereby reducing the volume of this expandable body 70. Accordingly, the fit of the patient interface 16 on the patient 18 via the strap 62 is loosened and therefore the force of the patient interface 16 pressing against the skin of the patient 18 is reduced. Although the expandable body 70 was described before by comprising a valve that may be used to increase or decrease the volume of the expandable body 70, it goes without saying that a design wherein only a decrease of the volume of the expandable body 70 is possible lies also within the scope of the present invention. The fluid used to increase or decrease the expandable body 70 as mentioned above can be any suitable fluid that can be used for such a purpose. Not limiting examples for these fluids are air, water, oil, any gas or the like. Preferably, this fluid is a non-toxic fluid in order to avoid any danger or hazard to the patient 18. More preferably, this fluid is air since this fluid does not provide any danger to the patient if anything is damaged. Further, air is quite easily to be handled via a compressor if it is desired to increase the volume of the expandable body 70, for example to inflate a balloon. Also, the mere deflation of the expandable body 70 is unproblematic for the surrounding environment when air is used.

Although the adjustment mechanisms 64 and 66, here the electroactive polymer 68 and the expandable body 70 are shown within one embodiment of the patient interface 16, it goes without mentioning that also patient interfaces are possible wherein only one type of the described adjustment mechanisms is present. Also, any other combination of the afore-mentioned adjustment mechanisms, manual, via electric motors, electroactive polymers or expandable bodies, may be present within the same embodiment of a patient interface according to the present invention.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. Patient interface system, with
a patient interface for providing a patient with a flow of gas, with a headgear for attaching the patient interface to the patient, and at least one adjustment mechanism for regulation of a force for attaching the patient interface to the patient with the headgear, and
a detector for monitoring a degree of blood flow through at least one blood vessel of the patient, with at least one sensor and at least one monitoring unit,
wherein the at least one sensor is able to detect characteristic data of a blood flow through the at least one blood vessel and is connected to the at least one monitoring unit for transmitting the detected data to the at least one monitoring unit,
wherein the at least one monitoring unit is able to receive the data from the at least one sensor and is able to assess the degree of blood flow through the at least one blood vessel based on these data,
wherein the at least one monitoring unit is able to provide a signal based on the assessed blood flow through the at least one blood vessel, and
wherein the at least one adjustment mechanism is configured to regulate the force for attaching the patient interface to the patient with the headgear based on the signal provided by the detector.

2. Patient interface system of claim 1, wherein the monitoring unit is able to assess a degree of occlusion of the at least one blood vessel based on the assessed blood flow, and wherein the signal is based on the assessed degree of occlusion.

3. Patient interface system of claim 1, wherein the sensor comprises a light source for transmitting light into the skin and an optical sensor for detecting the reflected light, a sound detector or a pressure sensor.

4. Patient interface system of claim 1, further comprising an evaluation unit, wherein the evaluation unit is designed to receive and to process the signal from the monitoring unit and wherein the evaluation unit is able to control other devices via a control signal based on these processed signals.

5. Patient interface system of claim 1, wherein the patient interface comprises a cushion element, wherein the cushion element comprises the at least one sensor.

6. Patient interface system of claim 5, wherein the at least one sensor comprises a light source for transmitting light into the skin and an optical sensor for detecting the reflected light, a sound detector or a pressure sensor.

7. Patient interface system of claim 5,
wherein the at least one sensor is located in an area of the cushion element that corresponds to an area on the skin of the patient that is prone to a formation of red marks when the cushion element is applied to the patient.

8.-13. (canceled)

14. Method for preventing a formation of red marks by a patient interface on a skin of a patient, with the following steps:
attaching the patient interface to the patient by an attachment force, such that the patient interface contacts the skin of the patient,
assessing a degree of blood flow through at least one blood vessel of the patient where the patient interface contacts the skin, and
adjusting the attachment force such that a blood flow is present, preferably such that the blood flow is above a predetermined threshold.

15. Method of claim 14, wherein assessing the degree of blood flow is realized by detecting characteristic data of the blood flow through the at least one blood vessel of the patient.

16-18. (canceled)

19. Patient interface system of claim 1, wherein the detector is part of the patient interface.

20. Patient interface system of claim 1, wherein the sensor and the at least one monitoring unit are separated from each other, wherein the sensor is arranged at the patient interface, and wherein the at least one monitoring unit is arranged at an external device which is part of the patient interface system.

21. Patient interface system of claim 1, wherein the headgear comprises a strap, and wherein the at least one adjustment mechanism comprises an electric motor for adjusting the length of the strap.

22. Patient interface system of claim 1, wherein the headgear comprises a strap, and wherein the at least one adjustment mechanism comprises an electroactive polymer for adjusting the length of the strap.

23. Patient interface system of claim 1, wherein the headgear comprises a strap, and wherein the at least one adjustment mechanism comprises an expandable body that is in- or deflatable via a controllable valve for adjusting the length of the strap.

24. Patient interface system, with
a patient interface for providing a patient with a flow of gas, with a headgear for attaching the patient interface to the patient, and at least one adjustment mechanism for regulation of a force for attaching the patient interface to the patient with the headgear,
a detector for monitoring a degree of blood flow through at least one blood vessel of the patient, with at least one sensor, at least one monitoring unit, and an evaluation unit, and
a sound generator,
wherein the at least one sensor is able to detect characteristic data of a blood flow through the at least one blood vessel and is connected to the at least one monitoring unit for transmitting the detected data to the at least one monitoring unit,
wherein the at least one monitoring unit is able to receive the data from the at least one sensor and is able to assess the degree of blood flow through the at least one blood vessel based on these data,
wherein the at least one monitoring unit is able to provide a signal based on the assessed blood flow through the at least one blood vessel,
wherein the evaluation unit is designed to receive and to process the signal from the monitoring unit, and
wherein the evaluation unit is configured to control the sound generator to produce an alarm if the signal indicates that a degree of blood flow within the at least one blood vessel or a degree of occlusion of the at least one blood vessel passes a predetermined threshold, said alarm indicating the patient that the attachment force of the patient interface is too strong and that the force has to be adapted by means of the at least one adjustment mechanism.
